(19) United States
(12) Patent Application Publication
(10) Pub. No.: US 2009/0259166 A1
(43) Pub. Date: Oct. 15, 2009

(54) Systems and Methods for Transdermal Photo-Polymerization

(75) Inventors: Robert K.G. Trow, Nanuet, NY (US); Andrew S. Thomas, New City, NY (US); Nathaniel David, Calabasas, CA (US)

Correspondence Address: WILSON, SONSINI, GOODRICH & ROSATI, 650 PAGE MILL ROAD, PALO ALTO, CA, 94304-1050, US

(73) Assignee: Kythera Biopharmaceuticals, Inc., Calabasas, CA (US)

(21) Appl. No.: 12/101,357

(22) Filed: Apr. 11, 2008

(51) Int. Cl. A61N 1/30; A61K 31/78

(52) U.S. Cl. 604/20; 424/78.31

(57) ABSTRACT

Systems and methods of polymerizing compositions with a light source may be provided. A device may include a handheld light source, which may include an LED, which may provide light at limited wavelengths to polymerize a composition. The device may be used to photo-polymerize dermal fillers transdermally via ex vivo applications. Dermal fillers may be compositions including PEODA 3400 and eosin Y, which may be polymerizable when exposed to light.

### CROSS-REFERENCE

This application claims priority to United Kingdom Application No. (Not yet Assigned) filed on Apr. 10, 2008 entitled Systems And Methods For Transdermal Photo-Polymerization which is incorporated herein by reference in its entirety, and to which application we claim priority under 35 USC 119(a).

### FIELD

The invention is directed to systems and methods of photo-polymerizing dermal fillers. The invention includes dermal filler formation, a light source to irradiate and polymerize such dermal fillers, and methods for using the same.

### BACKGROUND OF THE INVENTION

Various cosmetic surgery techniques include injecting dermal fillers. There may be various compositions and methods for administering dermal fillers, and devices emitting light have been used to polymerize dermal fillers, which require a certain intensity of light to penetrate the skin. However, previous devices have been emitting a broad spectrum of light, which included emitting damaging UV radiation as well as burning infrared radiation. Such devices can cause burns and other damage to exposed skin. In addition, such devices are costly and are often large and complex to use.

There is a need for improved systems and methods for photo-polymerizing dermal fillers that eliminate the potentially adverse effects of previous devices and that are relatively simple to use. There is a further need for dermal filler compositions that may polymerize at non-harmful wavelengths of light.

### SUMMARY OF THE INVENTION

The invention provides systems and methods of photo-polymerizing dermal fillers. Various aspects of the invention described herein may be applied to any of the particular applications set forth below or for any other types of light emitting devices and methods. The invention may be applied as a standalone system or method, or as part of an integrated method for polymerizing materials. It shall be understood that different aspects of the invention can be appreciated individually, collectively, or in combination with each other.

One aspect of the invention provides a light emitting device that may emit light at a limited wavelength. In one embodiment of the invention, the device comprises one or more light emitting diodes (LED) that may emit light with wavelengths tuned to a wavelength that causes photo-polymerization of a photo-polymerizable material. The light emitting device may comprise a portable unit which may be connected to a handheld light source through an umbilicus. The light emitting device may be less costly and easier to use than other larger, more complex and more costly devices.

In another aspect, the invention provides a method for polymerizing a dermal filler composition. A dermal filler composition may be administered subdermally to a soft tissue region. A light may be administered transdermally to the dermal filler composition where the light may have a wavelengths tuned to the polymerization wavelength of the dermal filler composition. By having a limited light emission spectrum, the invention may advantageously eliminate potential adverse effects such as UV exposure or burns. In one embodiment of the invention, the light source may be immediately adjacent to the soft tissue region with the dermal filler composition. In another embodiment of the invention, the light may be administered to the dermal filler composition for a relatively short period of time, which may advantageously allow ease of use.

A dermal filler composition may comprise PEODA 3400, modified hyaluronic acid, triethanolamine, and N-vinyl pyrrolidone in accordance with another aspect of the invention. The dermal filler composition may further comprise eosin Y. In another embodiment of the invention, the dermal filler composition may comprise PEODA 3400, modified hyaluronic acid, and triethanolamine where the ratio between PEODA 3400 and the modified hyaluronic acid is greater than 1:1. The dermal filler composition may further comprise eosin Y.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### Incorporation By Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows absorption of eosin Y for varying wavelengths.

FIG. 2 shows a light emitting device with a handheld light source.

FIG. 3A shows photofiller at 0 seconds of light exposure in vitro.

FIG. 3B shows photofiller at 20 seconds of light exposure in vitro.

FIG. 4A shows photofiller at 0 seconds of transdermal light exposure in vivo.

FIG. 4B shows photofiller at 20 seconds of transdermal light exposure in vivo.

FIG. 5A shows photofiller at 0 seconds of transdermal light exposure in vivo using a light emitting device with a higher wattage capacitor.

FIG. 5B shows photofiller at 5 seconds of transdermal light exposure in vivo using a light emitting device with a higher wattage capacitor.

FIG. 5C shows photofiller at 10 seconds of transdermal light exposure using a light emitting device with a higher wattage capacitor.

FIG. 6 shows rat skin with implanted photofiller at 15 seconds of exposure.

FIG. 7 shows photofiller at 15 seconds of exposure after removed from the rat.

FIG. 8A shows 10% static compression moduli for photofiller as exposed to varying times of a light emitting device using an LED.

FIG. 8B shows 10% static compression moduli for photofiller as exposed to varying numbers of IPL flashes.

### DETAILED DESCRIPTION OF THE INVENTION

The invention provides a light-emitting device that may emit light only from a narrow portion of the electromagnetic spectrum. For example, a device may emit substantially only light with wavelengths that trigger polymerization of a photo-polymerizable material or only light with wavelengths that trigger polymerization of a photo-polymerizable material. Such polymerizable material may contain eosin or eosin Y, which may result in the material polymerizing at wavelengths where absorption of eosin is greatest.

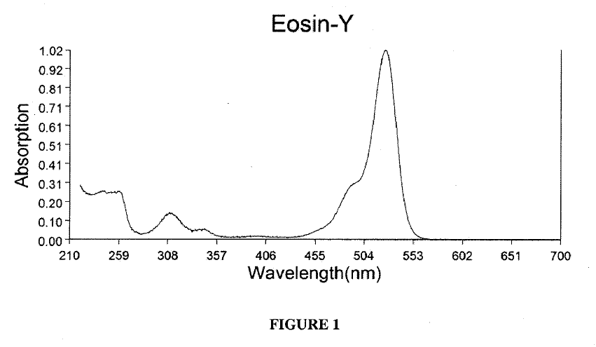

FIG. 1 shows absorption of eosin Y for varying wavelengths. For eosin Y, a peak absorption may occur at around 524 nm with a spread of the entire absorption spectrum of ±59 nm. In a preferable embodiment of the invention, the device may emit light with wavelengths exclusively between 470 and 590 nm or between 520 and 540 nm. In another embodiment the device may emit light with wavelengths between 500 and 550 nm. In yet another embodiment of the invention, the device may emit light with wavelengths 520 nm or longer. Alternatively, the device may emit light at a limited wavelength where a limited wavelength includes a more limited spectrum than a broad spectrum, such as the broad spectrum emitted by an Intense Pulsed Light device.

The device may emit light such that a peak intensity of emission is at a wavelength above 520 nm but not less than 520 nm. Alternatively, the device may emit light such that a peak intensity of emission is at a wavelength above 500 nm but not less than 500 nm, or above 510 nm but not less than 510 nm, or above 530 nm but not less than 530 nm, or above 540 nm but not less than 540 nm, or above 550 nm but not less than 550 nm, or above 600 nm but not less than 600 nm.

The light-emitting device may emit light such that the light only has a single peak intensity. The single peak intensity of emission may beat a wavelength above 510 nm but not less than 510 nm. Similarly, the single peak intensity of emission may be at a wavelength above 490 nm but not less than 490 nm, above 500 nm but not less than 500 nm, above 520 nm but not less than 520 nm, above 550 nm but not less than 550 nm, above 600 nm but not less than 600 nm.

In some embodiments, the device may emit light such that >80%, >90%, >95%, >96%, >97%, >98%, >99%, >99.9%, or >99.99% of the photons emitted are at a wavelength between 480 and 600 nm, or at a wavelength greater than 490, 500, 510, or 520 nm. The device may also emit light such that <0.01%, <0.1%, <1%, <2%, <3%, <4%, <5%, <10%, or <20% of the photons emitted are at a wavelength shorter than 480 nm or longer than 600 nm. The device may also emit light such that <0.01%, <0.1%, <1%, <2%, <3%, <4%, <5%, <10%, or <20% of the photons emitted are at a wavelength longer than 600, 610, 620 or 630 nm.

The light emitted by the device may be such that a peak intensity of emission is within 5 nm of the peak absorption of the excitable fluorophor within a dermal filler. Alternatively, the peak intensity of emission may be within 0.01 nm, 0.1 nm, 1 nm, 2 nm, 3 nm, 4 nm, 5 nm, 7 nm, 10 nm, 15 nm, 50 nm, or 100 nm of the peak absorption of the excitable fluorophor within the dermal filler. In some embodiments of the invention, the excitable fluorophor may be eosin Y.

The light emitted by the device may include a combination of features. For example, the device may emit a light such that a peak intensity of emission is at a wavelength above 520 nm but not less than 520 nm and such that >95%, >96%, >97%, >98%, >99%, >99.9%, or >99.99% of the photons emitted are at a wavelength between 520 and 600 nm. The device may also emit a light such that a peak intensity of emission is at a wavelength above 520 nm but not less than 520 nm and such that <0.01%, <0.1%, <1%, <2%, <3%, <4% or <5% of the photons emitted are at a wavelength longer than 600, 610, 620 or 630 nm. A light emitted by the device may have a single peak intensity of emission such that the single peak intensity is at a wavelength above 510 nm but not less than 510 nm and the light may be such that >95%>96%, >97%, >98%, >99%, >99.9%, or >99.99% of the photons emitted are at a wavelength between 510 and 600 nm. The light emitted by the device may also have a single peak intensity of emission such that the single peak intensity is at a wavelength above 510 nm but not less than 510 nm and the light emitted may be such that <0.01%, <0.1%, <1%, <2%, <3%, <4% or <5% of the photons emitted are at a wavelength longer than 600, 610, 620 or 630 nm.

The light emitted by the light emitting device may be such that the peak intensity of emission is within 1 nm, 2 nm, 3 nm, 4 nm, 5 nm, 50 nm, or 100 nm of the peak absorption of the excitable fluorophor within a dermal filler and such that >95% of the photons emitted are at a wavelength between 480 and 600 nm. Similarly, the light emitted by the light emitting device may be such that the peak intensity of emission is within 1 nm, 2 nm, 3 nm, 4 nm, 5 nm, 50 nm, or 100 nm of the peak absorption of the excitable fluorophor within a dermal filler and such that <5% of the photons emitted are at a wavelength shorter than 480 nm or longer than 600 nm. The fluorophor is preferably eosin Y.

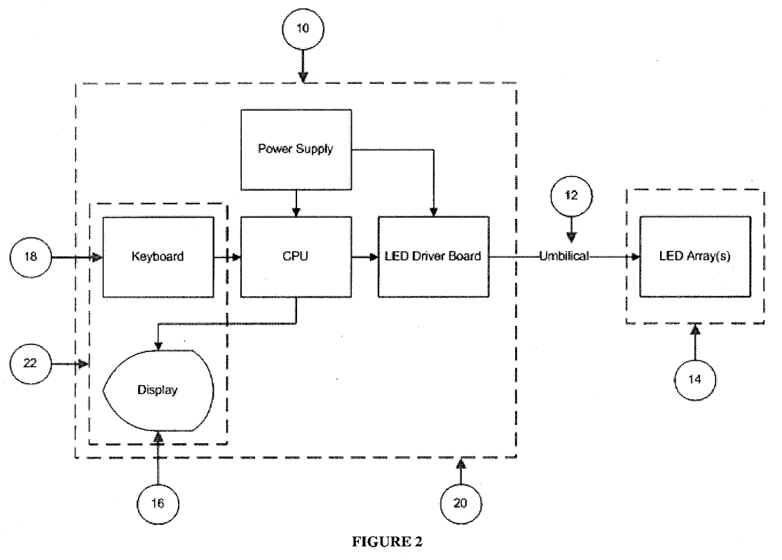

FIG. 2 shows a light emitting device with a handheld light source 14 in accordance with one aspect of the invention. The light emitting device comprises one or more light emitting diodes (LEDs). The LEDs can be part of a portable unit 10 or on a handheld device 14. In one implementation, there may be two LEDs. However, it is to be understood that, in practice, an arrangement of one or more LEDs can be used. In one implementation, such LEDs may be commonly actuated and operated as a group. For example, a single set of controls may turn the LEDs on or off together.

The light emitting device may comprise a portable unit 10 which may be connected to a handheld light source 14 through an umbilicus 12. In the alternative, the handheld light source 14 may not be directly connected to the unit 10, but may communicate with the unit wirelessly. The portable unit 10 can be consigned for placement on a tabletop or desktop. In a preferable implementation, the portable unit 10 weighs less than 15 pounds. The portable unit 10 may include a cradle for the handheld light source 14 to rest when not being used.

The handheld light source has a cross-sectional area from which the light may emit. The cross-sectional area may be at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, or 10.0 square cm. In some embodiments, the cross-sectional area is between 2-20, 3-10, or 4-5 square cm. The cross-sectional area may have any number of shapes or arrangements, such as a rectangular cross-sectional area. The handheld light source 14 may also have various shapes and configurations, such as having a gun-like shape or a wand-like shape.

The light source may be controlled by a control unit 20 which may be part of the portable unit 10. The control unit 20 may permit a program controlled actuation in which factors such as the intensity of the light or time of light emitted may be programmed. In one example, the light control unit may be pre-programmed. Alternatively, the light control may be pre-programmed but may allow variation in the program by a user, such as in situations where it may be possible to provide an accommodation of the program to the parameters to which the polymerizable material may be applied. Similarly, a control unit may have multiple programs for light emission. For example, depending on the amount of polymerizable composition or location of the composition, a user may select a program that fits a situation. In one example, the light control may be controlled directly by a user. For example, a user may adjust the intensity of a light with a knob while the device is operating.

The control unit 20 may be connected to the light source 14 or be part of the portable unit 10. The control unit 20 may include some sort of user interface or control panel 22. For example, a control panel 22 may include means 18 for adjusting light intensity or timing of light emitted, such as buttons, switches, touchscreens, keys, knobs, and other input-output communication devices 18. A control panel 22 may also include a display 16 to show a user information relating to the light to be emitted. A control panel may also include preset means which may allow a user to select a program of the control device for particular situations.

The light source 14 may be programmable to emit light at multiple intensities. For example, a light source may emit light at intensities that range from 0 to 5 mW, 0 to 10 mW, 0 to 15 mW, 0 to 20 mW, 0 to 25 mW, 0 to 30 mW, 0 to 50 mW, or 0 to 100 mW. The light source may also be programmable to emit lights for different times. For example, a light source may be programmed to emit light for a programmed length of time. For example, the light source can be programmed to emit light for up to 999 seconds, 500 seconds, 100 seconds, 10 seconds, 5 seconds, 4 seconds, 3 seconds, 2 seconds, or 1 second. A light source may also be programmed to emit light at various times. For example, a light source may cycle through different light exposing times, such as turning on for 5 seconds, then turning off for 5 seconds, and repeating. A light source may also emit light at various times at various intensities. For example, a user may program the light source to emit a light for 5 seconds at 5 mW, then turn off for 3 seconds, then turn on for 10 seconds and emit light at 20 mW, and so forth. Programming the light source may include pre-programmed settings, pre-programmed settings plus user control, or direct user control.

The devices herein may have varying power outputs. In one embodiment, a device has an output power of up to 1 W. The device may also run on 110 V AC single phase in one implementation. The device may also contain additional features such as self-contained cooling. In some instances, a device can run on one or more batteries.

Light emitting devices such as those described herein can be used for augmenting a region of interest or polymerizing a photo-polymerizable material that has been delivered transdermally or subcutaneously. The method comprises the steps of administering subdermally to a region of interest a dermal filler composition; and applying light transdermally to the region using the light emitting devices described herein. The light preferably is from an LED source. The transdermal application of light is preferably made ex vivo (external to body) as opposed to in vivo (using a catheter to bring light under skin). The region of interest can be any region including bone fractions, bone joints, and soft tissue. Preferably, light is administered externally to a soft tissue region after a dermal filler has been applied subdermally to the region to augment its appearance.

A light may also be administered transdermally to the dermal filler composition where the light has wavelength(s) tuned to the polymerization wavelength of the dermal filler composition. For example, a dermal filler composition can contain eosin, which results in the material polymerizing at wavelengths where absorption of eosin is greatest, e.g., 520-540 nm. In one embodiment, the light may be administered to the dermal filler composition ex vivo (without the use of a catheter) and may have wavelengths between 520 and 540 nm. In another implementation, the light may be administered transdermally to the soft tissue region where the dermal filler composition has been administered and have wavelengths between 500 nm and 550 nm. In yet another implementation, the dermal filler composition may be transdermally exposed to light that may have wavelengths longer than 520 nm. The light may be administered to the dermal filler composition subdermally and have wavelengths between 500 nm and 550 nm.

In one example, the light may be administered by the previously described device with a handheld light source 14.

The light can be applied while immediately adjacent to the soft tissue region where a dermal filler composition has been injected. For instance, a handheld light source 14 can be held directly next to skin into which a dermal filler composition has been injected or administered. Alternatively, a light can be administered from a handheld light source 14 transdermally from a distance away from the skin into which a dermal filler composition has been injected. Such distance can be, for example, at least 5, 10, or 20 cm. The light used to activate polymerization can be delivered transdermally.

The light can be administered to the soft tissue region into which a dermal filler composition has been injected at varying intensities and for different periods of time. In one embodiment of the invention, the light is administered with an intensity that ranges from 0 to 25 mW, 3 to 20 mW, or 4 to 10 mW. In some embodiments of the invention, the light is administered at an intensity of greater than 1 mW, 2 mW, 3 mW, 4 mW, 5 mW, 6 mW, 7 mW, 8 mW, 9 mW, 10 mW, 11 mW, 12 mW, 13 mW, 14 mW, 15 mW, 16 mW, 17 mW, 18 mW, 19 mW, 20 mW, 21 mW, 22 mW, 23 mW, 24 mW, or 25 mW. The light exposure may occur for a relatively short period of time, such as up to 1 second, 2 seconds, 3 seconds, 4 seconds, 5 seconds, 6 seconds, 7 seconds, 8 seconds, 9 seconds, 10 seconds, 11 seconds, or 12 seconds. In one embodiment, a dermal filler composition is exposed to light for up to 15 seconds, 20 seconds, 30 seconds, 60 seconds, 120 seconds, or 999 seconds. In other embodiments, 2 to 18 seconds, 5 to about 15 seconds, about 7 to about 12 seconds, about 8 to about 10 seconds. Alternatively a dermal filler composition may be intermittently exposed to light for a period of time.

In some of the methods of the invention, a dermal filler composition comprises one or more cross-linkable polymeric or monomeric materials or derivatives thereof.

The polymeric or monomeric materials can optionally contain modified reactive groups that facilitate polymerization, attachment or cross-linking of the polymeric or monomeric material upon exposure to light. Thus upon exposure to a visible light, a liquid form of the cross-linkable polymeric or monomeric material or derivative thereof in the dermal filler composition takes on a gel form, and is amenable to desired contouring and manipulation. This results in a desired, solid and/or semisolid polymerized form in situ. The use of derivatized monomers or polymers can provide for more stable polymers and networks that are more resistant to biodegradation.

Virtually any polymeric material that may be modified to include a light-activated derivatized reactive group may be used as the cross-linkable material in the preparation of the present dermal filler composition. Either synthetic or natural monomers/polymers may be used.

Useful cross-linkable materials include, but are not limited to, poly(lactic acid) (PLA), poly(glycolic acid) (PGA), and their copolymers, poly(lactic-co-glycolic acid) (PLGA). In some embodiments, polyethylene glycol diacrylate monomers (“PEG-diacrylate”) are used as a starting point for selectively customizing the mechanical and persistence (durability) properties of the tissue augmentation material. Synthetic polymers include poly(ethylene oxide)(PEO) based polymers and can be found as copolymers such as Pluronic, a triblock copolymer of poly(ethylene oxide) and poly(propylene oxide) (PEO-PPO-PEO), or derivatized to be capable of photoinitiated cross-linking, such as poly(ethylene oxide)diacrylate (PEODA). In some embodiments, PEODA 3400 is used.

Other examples of useful polymers include, but are not limited to: polyalkylene oxides, polyethylene glycols, polyethylene oxides, partially and fully hydrolyzed polyvinylalcohols, poly(vinylpyrrolidone), poly(t-ethyloxazoline), poly(ethylene oxide)-co-poly(propylene oxide) block copolymers (poloxamers and meroxapols), polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid, polymethacrylic acid, poly(hydroxyethylmethacrylate), poly(hydroxyethylacrylate), poly(methylalkylsulfoxide methacrylate), poly(methylalkylsulfoxide acrylate), and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers such as polyethylene glycol/poly(N-isopropylacrylamide)thereof; polyoxazolines, including poly(methyloxazoline) and poly(ethyloxazoline); polyvinylamines, polyacrylamide (PAA), poloxamines, carboxymethyl cellulose, and hydroxyalkylated celluloses such as hydroxyl-ethyl cellulose and methylhydroxypropyl celluloses.

Natural monomers and polymers useful in the dermal filler compositions of the methods of the invention include glycosaminoglycans such as hyaluronic acid, chondroitin sulfate A, chondroitin sulfate C, dermatan sulfate, keratan sulfate, keratosulfate, chitin, chitosan, and derivatives thereof. Therefore, while not exhaustive, examples of natural monomers or polymers which may be used in the dermal filler compositions in the methods of the invention include: polypeptides, polysaccharides or carbohydrates such as polysucrose, hyaluronic acid, dextran, heparin sulfate, chondroitin sulfate, heparin, or alginate, and proteins such as gelatin, collagen, albumin or ovalbumin or copolymers or blends thereof. Celluloses, including dextran and similar derivatives may also be used. Extracellular matrix proteins that can be used include those, such as collagens, elastins, laminins, gelatins, and fibronectins. Fibrin, a naturally occurring peptide important for its a role in wound repair in the body, and alginate, a polysaccharide derived from seaweed containing repeating units of mannuronic and guluronic acid, may also be used. One may use various combinations of the above compound, and may include chemically modified forms, mimetics, or derivatives thereof. For proteins, one may use recombinant forms, analogs, forms containing amino acid mimetics, and other various protein or polypeptide-related compositions.

Any of the monomers and polymers of the present invention may be derivatized appropriately to provide cross-linking capability under preselected conditions. A functional group or a moiety capable of mediating formation of a polymer or network can be added to a naturally occurring molecule or a synthetic molecule practicing methods known in the art. Functional groups include alkenyl moieties such as acrylates, methacrylates, dimethacrylates, oligoacrylates, oligomethacrylates, ethacrylates, itaconates or acrylamides. Additional functional groups include aldehydes. Other functional groups may include ethylenically unsaturated monomers including, for example, alkyl esters of acrylic or methacrylic acid such as methyl methacrylate, ethyl methacrylate, butyl methacrylate, ethyl acrylate, butyl acrylate, hexyl acrylate, n-octyl acrylate, lauryl methacrylate, 2-ethylhexyl methacrylate, nonyl acrylate, benzyl methacrylate, hydroxyalkyl esters of the same acids such as 2-hydroxyethyl acrylate, 2-hydroxyethyl methacrylate, and 2-hydroxypropyl methacrylate, the nitrile and amides of the same acids such as acrylonitrile, methacrylonitrile, and methacrylamide, vinyl acetate, vinyl propionate, vinylidene chloride, vinyl chloride, and vinyl aromatic compounds such as styrene, t-butyl styrene and vinyl toluene, dialkyl maleates, dialkyl itaconates, dialkyl methylene-malonates, isoprene and butadiene. Suitable ethylenically unsaturated monomers containing carboxylic acid groups include acrylic monomers such as acrylic acid, methacrylic acid, ethacrylic acid, itaconic acid, maleic acid, fumaric acid, monoalkyl itaconate including monomethyl itaconate, monoethyl itaconate, and monobutyl itaconate, monoalkyl maleate including monomethyl maleate, monoethyl maleate, and monobutyl maleate, citraconic acid and styrene carboxylic acid. Suitable polyethylenically unsaturated monomers include butadiene, isoprene, allylmethacrylate, diacrylates of alkyl diols such as butanediol diacrylate and hexanediol diacrylate, divinyl benzene and the like.

By way of example, and not limitation, and in particular embodiments, the polymer can comprise synthetic reactants and comprises poly(ethylene glycol) (PEG) or a derivative thereof. In some embodiments, the polymer derivative comprises poly(ethylene oxide)diacrylate (PEODA) or poly(ethylene glycol)diacrylate (PEGDA).

A combination of more than one synthetic or natural monomer/polymer as described above can be used in the dermal filler compositions. For example, in various embodiments, PEODA is used in combination with a polysaccharide (monomer or polymer) such hyaluronic acid, chrondroiten sulfate, or alginate. In some of the embodiments, the polysaccharide (monomer or polymer) is modified such that it is also capable of cross-linking. In some embodiments, the modified polysaccharide (monomer or polymer) is at least partially cross-linked prior to combining with the PEODA in the dermal filler composition. In one embodiment, the at least partially cross-linked modified polysaccharide (monomer or polymer) is capable of cross-linking with itself or with the PEODA upon administration of light in the methods of the invention. In other embodiments, the at least partially cross-linked modified polysaccharide (monomer or polymer) is incapable of cross-linking with the PEODA upon administration of light in the methods of the invention. The modified polysaccharide described herein can be modified hyaluronic acid.

A dermal filler composition can optionally further comprise a photoinitiator, such as eosin Y. Photoinitiator moieties are long-wave ultra violet (LWUV) light-activatable molecules. Examples of photoinitiators include, e.g., 4-benzoylbenzoic acid, [(9-oxo-2-thioxanthanyl)-oxy]acetic acid, 2-hydroxy thioxanthone, and vinyloxymethylbenzoin methyl ether; visible light activatable molecules such as acridine orange, ethyl eosin, eosin Y, eosin B, erythrosine, fluorescein, methylene green, methylene blue, phloxime, riboflavin, rose bengal, thionine, and xanthine dyes, and thermally activatable molecules such as 4,4′azobis(4-cyanopentanoic)acid and 2,2-azobis[2-(2-imidazolin-2-yl)propane]dihydrochloride.

A dermal filler composition can optionally further comprise a co-catalyst. Examples of a suitable co-catalysts include amines, such as a tertiary amine, e.g., triethanolamine, N-methyl diethanolamine, triethylamine, dibenzylamine, NN-dimethyl benzylamine, dibenzyl amine, N-benzyl ethanolamine, or N-isopropyl benzylamine.

A dermal filler composition can optionally also comprise an accelerant. Examples of accelerants include N-vinyl pyrrolidone, 2-vinyl pyridine, 1-vinyl imidazole, 9-vinyl carbazole, acrylic acid and 2-allyl,2-methyl,1-3-cyclopentane dione. The accelerant used may be selected in part based on the initiator, coinitiator, polymers monomers used in the dermal filler, and time it takes the composition to polymerize and harden.

In one embodiment, a dermal filler composition comprises PEODA 3400, modified hyaluronic acid, triethanolamine, and N-vinyl pyrrolidone. Such composition may further comprise eosin or eosin Y. In another embodiment of the invention, the dermal filler composition comprises PEODA 3400, modified hyaluronic acid, and triethanolamine where the ratio between PEODA 3400 and the modified hyaluronic acid is greater than 1:1. The ratio between PEODA 3400 and modified hyaluronic acid may also be greater than 2:1, 3:1, 5:1, or 10:1.

The dermal filler composition comprises a combination of a PEODA/Restylene® (a commercially available modified hyaluronic acid, U.S. Pat. No. 5,827,937) solution. In some embodiments, the PEODA/Restylene® solution comprises up to 10%, 15%, 20%, 30%, 40%, 50%, 60%, 70%, or even up to 80% PEODA (w/w, v/w, or w/v). The dermal filler composition may comprise modified hyaluronic acid, which may have concentrations of up to 10%, 20%, 30%, 40%, 50%, 60%, 70%, 80% or 90% (w/w, v/w, or w/v). The dermal filler composition may comprise triethanolamine, which may have concentrations of up to 10%, 20%, 30%, 40%, 50%, 60%, 70%, 80% or 90% (w/w, v/w, or w/v). The dermal filler composition may comprise N-vinyl pyrrolidone, which may have concentrations of up to 10%, 20%, 30%, 40%, 50%, 60%, 70%, 80% or 90% (w/w, v/w, or w/v).

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLES

### Example 1

A dermal filler may be prepared in the following manner. An initiator solution may be prepared by dissolving 250 μg of eosin Y into 1 ml of NVP solution and vortexing until dissolved. PEGDA solution may be prepared by dissolving 200 mg of PEGDA in 100 μl of the initiator solution and 100 μl of PBS. The PEGDA solution may be used the same day.

Sixty μl of TEA may be added into 200 μl of the PEGDA solution. This may be a very viscous solution and may need to be pipetted very slowly and carefully. Then 40 μl of initiator solution may be added. This combination may be mixed on a vortex until uniformly pink throughout and is somewhat transparent pink with no particles of PEODA 3400. The material may be spun in a centrifuge to remove air bubbles, which may make it difficult to determine if the solution is well mixed. If particles are present, the material may be vortexed and then spun again in a centrifuge.

The photofiller solution may be stored in a foil covered tube to avoid ambient light exposure whenever possible during mixing procedure.

### Example 2

A light emitting device was assessed for ease of use and was tested for ability to polymerize a photofiller by varying lengths of time ranging from 5 to 30 seconds. The light emitting device is a very light compact device with a handheld LED-based light source which may be simple and easy to operate. The light-emitting device may weigh under 15 pounds and be less than 12 inches on a side. Preferably, it does not produce heating effects even when illuminating human skin for long periods of time. It was extremely efficient at inducing photo-polymerization of photofiller in vitro as well as transdermally in a mold and in vivo after intradermal transplantation. In addition, the device did not produce any detectable heating of the skin, even when placed against the user's forearm for more than a minute with constant full-power illumination.

The light-emitting device was tested for its ability to induce photo-polymerization of photofiller in a mold in vitro, transdermally in a mold, and after injection into rat dermis.

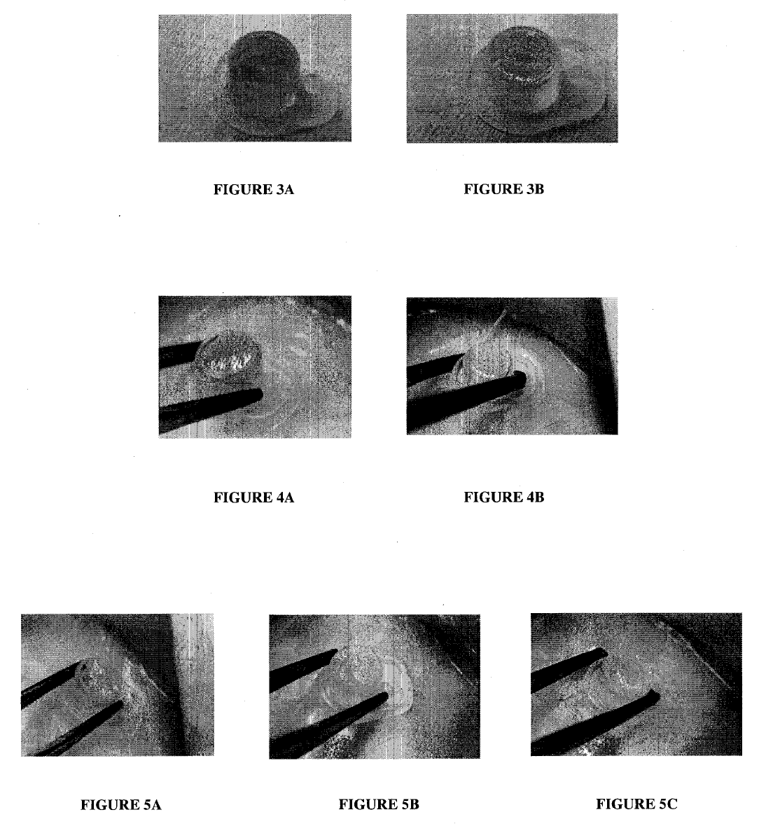

In vitro. A 100 μl aliquot of photofiller was placed into an inverted Epindorph tube cap (from a 0.5 ml tube) and then the handheld light source was held immediately adjacent to the material and the light-emitting device turned on for specified periods. In vitro polymerization was achieved after 10 and 20 seconds. FIG. 3A shows the photofiller at 0 seconds, and FIG. 3B shows the photofiller at 20 seconds. The photofiller was determined to have polymerized by ejecting the material from the cap and observing it to retain the shape of the mold. In addition, the ejected material was palpated to determine that it was firm. In contrast, the un-polymerized material could not be ejected from the mold as a structurally intact shape, as the un-polymerized material had a liquid, ‘honey-like’ consistency.

In vivo. The light-emitting device was also used to polymerize photofiller implants after placement of the photofiller material beneath the entire skin anatomy of rats. One hundred (100) μl photofiller implants were placed within Epindorph tube caps (see FIGS. 4A and 4B) and these taps were placed (open-side-up) beneath a rat skin flap such that the entire rat skin anatomy could be placed over the photofiller sample. The intention was to determine if the photofiller could still be polymerized by the light-emitting device despite the presence of the entire rat skin anatomy between the photofiller material and the light-emitting device. The samples (placed beneath the skin within the Epindorph caps) were illuminated immediately and their appearance and mechanical properties after polymerization through rat skin were assessed. Polymerization was achieved after 20 seconds through rat skin with the handheld light source (65 mW as measured with a radiometer at the edge of the hand piece) held immediately adjacent to skin with the skin flap covering the photofiller material within the mold. FIG. 4A shows the photofiller at 0 seconds of transdermal light exposure, and FIG. 4B shows the photofiller at 20 seconds of transdermal light exposure (65 mW). The implants were removed from their molds to mechanically confirm polymerization by palpation.

Higher Power LED. In some limited pilot testing, a higher power LED was adapted and tested at various lengths of time for polymerization of photofiller. A light-emitting device with a higher power may include a LED able to emit light with a power output of up to 2 W. Transdermal photo-polymerization was achieved very rapidly using the 2 W light-emitting device. Photofiller material was partially polymerized within 5 seconds and completely polymerized within 10 seconds, FIG. 5A shows the photofiller before transdermal light exposure with the 2 W light-emitting device. FIG. 5B shows the photofiller after 5 seconds of transdermal light exposure using the 2 W light-emitting device. FIG. 5C shows the photofiller at 10 seconds of transdermal light exposure using the 2 W light-emitting device.

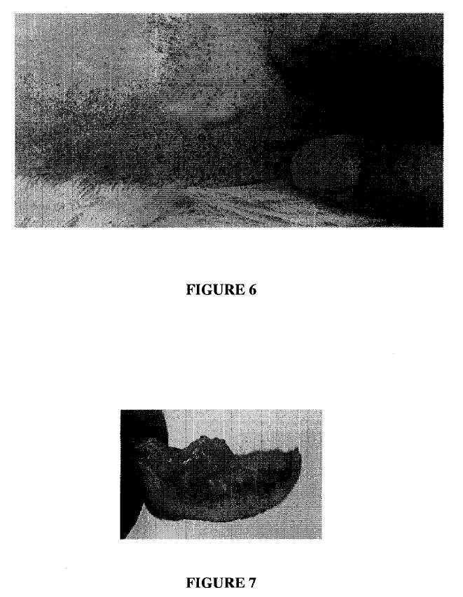

Trailing injection. Implants of photofiller were produced in rat skin using either 100 or 200 μl of photofiller material. A trailing injection (i.e., an injection in which the needle is inserted to a target depth and then the injectate is gradually delivered as the needle is slowly removed) may be used. FIG. 6 shows rat skin with implanted photofiller from a 100 μl trailing injection followed by 15 seconds of transdermal illumination with the 250 mW light-emitting device. FIG. 7 shows that photofiller implant resected from the rat skin.

### Example 3

An LED-based device and an Intense Pulsed Light (IPL) device were compared for their ability to polymerize samples of photofiller material. The intent of this experiment was to compare the ability of an LED-based device (which emits only a narrow portion of electromagnetic spectrum) to the ability of an IPL device (which emits a broad range of the electromagnetic spectrum and is quite damaging to skin) to induce photofiller polymerization. The comparison was performed as follows.

A photofiller solution was prepared with the following recipe. An initiator solution was made by dissolving eosin Y disodium salt (Sigma-Aldrich CAT# 45235)) in PBS (1.375 μg/mL eosin Y). 100 mg PEODA (3.4 KD MW SunBio CAT# P2AC-3)) was dissolved in 50 μL of initiator solution, 30 μL of PBS, and 20 μL of N-vinyl pyrrolidone (Sigma-Aldrich CAT# 95060). Final solutions were prepared by mixing this PEODA solution with 30 μL of triethanolamine (Sigma-Aldrich #90278) and 1 mL of PBS.

100 μL of this solution was polymerized in plastic mold (the caps of 0.5 mL microcentrifuge tubes). The IPL-mediated polymerization was initiated by exposure of the photofiller samples to three (3), twelve (12), or twenty four (24) 50 millisecond pulses from the Sciton IPL system at 5 J/cm . The LED-mediated polymerization was initiated by continuous exposure of the samples to 10, 15, 30, 60, and 120 seconds of LED emission (at 65 mW).

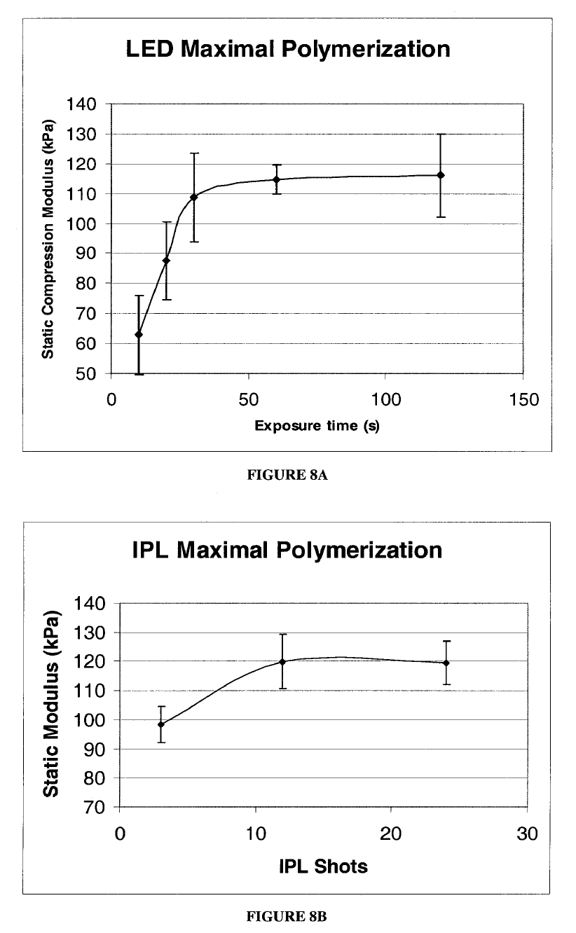

Measuring degree of cross-linking by static compression. Polymerized gels were then subjected to 10% static compression tests at 1 mm/min loading rate. Stress and strain were calculated, after which the compressive modulus was determined from the slope of the linear portion of the curve. FIG. 8A shows 10% static compression moduli for photofiller hydrogels as exposed to varying times of the maximum power light-emitting device using an LED.

FIG. 8B shows 10% static compression moduli for photofiller hydrogels as exposed to varying numbers of IPL flashes.

Measuring light intensity. Dorsal skin from a rat of approximately 1.44 mm in thickness was placed over the entire surface of a visible light pyranometer sensor (PMA 2144, Solar Light, Glenside, Pa.). Pyranometer measurements were all confirmed to be zero without LED exposure. Radiometric (i.e., light intensity) measurements were recorded using the highest theoretical output (250 mW) of the light-emitting device. The intensity of the light-emitting device at its highest setting was approximately 33 mW/cm² as measured by the pyranometric radiometric sensor. Intensity measurements over various parts of the skin averaged approximately 13 mW/cm². This data indicates that approximately 40% of light was detected through 1.44 mm skin.

For 100 μL of the polymer photofiller solution, a photofiller may have maximum polymerization when exposed to the light-emitting device set at 250 mW (i.e., which corresponds to approximately 65 mW of measurable emission as determined by radiometry at the edge of the hand piece) for 60 seconds in vitro. For 100 μL of the polymer photofiller solution, a photofiller may have maximum polymerization when exposed to the light-emitting device at 250 mW (i.e., 65 mW measurable) for 120 seconds for the dermis of a rat in vivo.

### Example 4

For augmentation of a bottom lip, a patient may be injected subdermally at a desired site of augmentation a 0.1 cc volume of a formulation comprising of: eosin Y, PEODA 3400, modified hyaluronic acid, triethanolamine, N-vinyl pyrrolidone. The site of augmentation is then illuminated for 60 seconds using a handheld light source (emitting 65 mW of light) which connected to-a portable device which include at least 1 LED.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-14. (canceled)

15. A method for polymerizing a dermal filler composition in situ comprising:
administering subdermally the dermal filler composition to a tissue region; and
applying external to the tissue region:
(a) a light-emitting diode; or
(b) a light having a peak intensity of emission at a wavelength greater than 520 nm but not less than 520 nm.

16. A method for augmenting a tissue comprising:
administering to a tissue site a dermal filler composition comprising an accelerant; and
applying transdermally to the tissue site a light having a limited wavelength.

17. The method of claim 15 or claim 16 wherein said light is from a light-emitting diode and/or is a light having wavelength between 500 nm and 550 nm.

18. The method of claim 16 wherein said light is from a light-emitting diode and/or is a light having wavelength greater than 520 nm but not less than 520 nm.

19. The method of any preceding claim wherein the light is applied ex vivo.

20. The method of claim 19 wherein said applying comprises (a) applying said light for up to 10 seconds; (b) is repeated at least twice.

21. The method of claim 15 wherein said dermal filler composition comprises an accelerant.

22. The method of claim 15 or claim 16 wherein said dermal filler composition additionally comprises PEODA 3400, eosin Y and/or N-vinyl pyrrolidone.

23. The method of any preceding claim wherein the light is applied by using a handheld light source.

24. The method of any preceding claim wherein the tissue region of interest is a soft tissue site.

25. An injectable, dermal filler composition, suitable for use in method claims 15 to 24, comprising:
PEODA 3400;
modified hyaluronic acid;
triethanolamine; and
N-vinyl pyrrolidone.

26. An injectable, dermal filler composition, suitable for use in method claims 15 to 24, comprising:
PEODA 3400;
modified hyaluronic acid; and
triethanolamine; wherein the ratio between said PEODA 3400 and said modified hyaluronic acid is greater than 1:1 w/w.

27. The composition of claim 25 or claim 26 further comprising eosin Y.
